(19) United States
(12) Patent Application Publication
(10) Pub. No.: US 2025/0177216 A1
(43) Pub. Date: Jun. 5, 2025

(54) DETACHABLE INSERT FOR SEPARATING TOPSHEET FROM DISPOSABLE ABSORBENT ARTICLE

(71) Applicant: Irene Richardson, El Paso, TX (US)

(72) Inventor: Irene Richardson, El Paso, TX (US)

(21) Appl. No.: 18/527,271

(22) Filed: Dec. 2, 2023

(51) Int. Cl. A61F 13/49; A61F 13/505

(52) U.S. Cl. CPC A61F 13/49007; A61F 13/505

(57) ABSTRACT

A disposable absorbent article utilized for absorption and containment of urine and other body exudates may incorporate components that may be detached from the article after use to provide disposal options based upon the user's preferences and circumstances. An absorbent insert positioned between a topsheet and a backsheet. The topsheet and insert may be releasably attached to the backsheet. An exposed edge is proximate the waist-side edge of the article, and the exposed edge is an opening allowing access to pull the insert by the insert end, protruding end, or tab to cause the topsheet to be pushed outward and detached from the article without the user directly touching or pulling the topsheet. The topsheet may be flushed down a toilet or septic tank, the insert may be composted, and the backsheet may be recycled; optionally, detachable components of the article may be discarded in a landfill or incinerated.

### FIELD

The present disclosure relates generally to the field of disposable absorbent articles utilized for the absorption and containment of urine and other body exudates, such as disposable diapers, disposable training pants, disposable inserts, disposable absorbent pads, disposable incontinence articles, and the like. More particularly, the disclosure relates to a disposable absorbent article that incorporates detachable components that may be separated from the article after use to provide disposal options for each of the detached components.

### BACKGROUND

Disposable articles for the absorption and containment of urine and other body exudates are generally known in the art. Such disposable articles have found particular utility in the fields of infant care, child care, and adult incontinency. Present commercially available disposable articles for such uses are comprised of a liquid permeable topsheet, a liquid impermeable backsheet, and disposed between the topsheet and the backsheet is an absorbent insert that encases the absorbent contents of the article. Disposable articles of this type effectively and efficiently absorb and contain urine or other body exudates. Such articles are designed for a single use and are simply discarded in their entirety along with human waste after use in a waste receptacle prior to being transported to a landfill. This ease and convenience of disposal has contributed to the increasing popularity of such disposable absorbent articles.

The vast majority of the disposable absorbent articles of the aforementioned type have been designed to be disposed of in solid waste landfills. Such articles are delivered to landfills in their entirety along with urine and fecal waste contained on the used disposable articles. The nondetachable design of these disposable articles have limited disposal options for consumers, and have not generally provided a sanitary means for the disposal of fecal material, such as by flushing the material down the toilet where it can be treated by the sewage system. This results in odor-causing fecal waste and bacteria being stored in the consumer's residence, child care centers, adult assisted living centers, or hospitals, prior to transporting the used disposable articles to the landfill. By directing used disposable articles to landfills, rather than to wastewater treatment facilities, fecal matter and bacteria may contaminate the groundwater in landfills and may spread diseases. Furthermore, the amount of disposable articles that have been discarded in landfills have led to the negative environmental impact that disposable articles have imposed on landfill capacity.

The relatively few products that have been designed specifically to be detached after use suffer from the following drawbacks. The removal of the topsheet requires for the user to have direct contact with the topsheet to handle and pull the topsheet, which is dirty and contains body waste. The removal of the absorbent insert does not cause the topsheet to be pushed out to be detached from the article. More specifically, to release the insert from the article, requires for the topsheet to be removed first. Tabs or grips used to facilitate detaching the topsheet are located on or attached to the topsheet, and are not located on or attached to the insert, and exposed edges are not used to allow the user to access the insert's tabs or grips in order to pull the insert from the interior of the article, and consequently, push the topsheet outward from the article to be detached.

### SUMMARY

In one embodiment of the present disclosure, a disposable absorbent article may comprise a liquid permeable topsheet that may be partially or completely detached from the article by the action of detaching the absorbent insert from the article. The detachable design of the article allows the user to select among several disposal options for each of the detached components of the article based upon the user's preferences and particular circumstances, such as flushing down a toilet or septic tank, composting, recycling, or disposing in a landfill. This provides the user with the option of directing the solid fecal waste and the soiled portion of the absorbent article to a wastewater treatment facility, rather than to a landfill. Consequently, this reduces or eliminates the amount of bacteria and odor-causing waste from the used disposable articles that must be stored by the user prior to disposing in the landfill, and reduces the potential for fecal matter and bacteria to contaminate the groundwater in landfills. This also mitigates the impact on the environment, since the disposable absorbent insert may be composted and the disposable backsheet may be recycled after use to reduce or potentially eliminate the amount of material and waste from the used disposable absorbent article that is delivered to the landfill.

The present disclosure was developed in order to remedy the previously-mentioned drawbacks associated with present disposable absorbent articles. This disclosure provides a disposable absorbent article that may be easily incorporated into the design of current disposable articles to produce a detachable absorbent article with components that may be flushed, composted, or recycled. Some of the benefits include: ease of use, sanitary for the user to maneuver, significant reduction or elimination of materials and bacteria disposed in the landfill, and disposal options for the consumer.

### BRIEF DESCRIPTION OF THE DRAWINGS

The foregoing and other objects, features and advantages of the present disclosure will become more apparent and better understood by reference to the following descriptions of non-limiting embodiments of the disclosure taken in conjunction with the accompanying drawings, wherein:

FIG. 1 is an exploded perspective view showing components of the disposable absorbent article.

FIG. 2 is a perspective view of an embodiment of the disposable absorbent insert.

FIG. 2A is a cross sectional view taken along line 2A-2A of FIG. 2.

FIG. 2B is a cross-sectional view taken along line 2B-2B of FIG. 2.

FIG. 3 is a top plan view in a stretched condition of one embodiment of the disposable absorbent article with hidden lines to show interior construction and having a corner folded back to show exterior construction.

FIG. 4 is a top plan view in a stretched condition of a second embodiment of the disposable absorbent article with hidden lines to show interior construction and having a corner folded back to show exterior construction.

FIG. 5 is a top plan view in a stretched condition of a third embodiment of the disposable absorbent article with hidden lines to show interior construction and having a corner folded back to show exterior construction.

FIG. 6A is a cross-sectional view taken along line 6A-6A of FIGS. 3, 5, 10, and 13.

FIG. 6B is a cross-sectional view taken along line 6B-6B of FIGS. 4 and 13.

FIG. 6C is a cross-sectional view taken along line 6C-6C of FIGS. 4 and 13.

FIG. 7A is a cross-sectional view taken along line 7A-7A of FIGS. 3 and 4.

FIG. 7B is a cross-sectional view taken along line 7B-7B of FIG. 5.

FIG. 8 is a perspective view demonstrating an initial step in the detachment of the absorbent article.

FIG. 9 is a perspective view demonstrating a further step in the detachment of the disposable absorbent article.

FIG. 10 is a top plan view in a stretched condition of a fourth embodiment of the disposable absorbent article with hidden lines to show interior construction and having a corner folded back to show exterior construction.

FIG. 11A is a cross-sectional view taken along line 11A-11A of FIG. 10.

FIG. 11B is a perspective view of an embodiment of the disposable absorbent insert of FIG. 10.

FIG. 12A is a cross-sectional view taken along line 12A-12A of FIG. 10.

FIG. 12B is a perspective view of a second embodiment of the disposable absorbent insert of FIG. 10.

FIG. 13 is a top plan view in a stretched condition of a fifth embodiment of the disposable absorbent article with hidden lines to show interior construction.

FIG. 14A is a cross-sectional view taken along line 14A-14A of FIG. 13.

FIG. 14B is a perspective view of an embodiment of the disposable absorbent insert of FIG. 13.

FIG. 15A is a cross-sectional view taken along line 15A-15A of FIG. 13.

FIG. 15B is a perspective view of a second embodiment of the disposable absorbent insert of FIG. 13.

### DETAILED DESCRIPTION

Various non-limiting embodiments of the present disclosure will now be described to provide an overall understanding of the principles of the structure, function, use, and disposal options of the disposable absorbent articles. One or more examples of non-limiting embodiments of the present disclosure will be further illustrated in the description below and in the Figures. Those of ordinary skill in the art will understand that the features illustrated or described in connection with one non-limiting embodiment may be combined with other features of other non-limiting embodiments. Further, alternative absorbent materials may be substituted for those described herein. Such modifications and variations are intended to be included within the scope of the present disclosure. Nothing in this description should be, however, considered limiting the scope of the claims.

The disposable absorbent article of the present disclosure is utilized for absorption and containment of urine and other body exudates, such as disposable diapers, disposable training pants, disposable inserts, disposable absorbent pads, disposable incontinence articles, and the like, which are used by infants, children, and adults. The detachable design of the article allows the user to select among several disposal options for each of the detached components of the disposable absorbent article based upon the user's preferences and particular circumstances, such as flushing down a toilet or septic tank, composting, recycling, or disposing in a landfill. For simplicity, the disposable absorbent article of this disclosure may be referred to as the article.

The topsheet or a portion of the topsheet may be flushed down the toilet. The absorbent insert may be composted or recycled where suitable accommodations or equipped facilities are available. The backsheet may be recycled where suitable accommodations or equipped facilities are available. The topsheet, absorbent insert, and backsheet may also be discarded at a conventional landfill site, or incinerated.

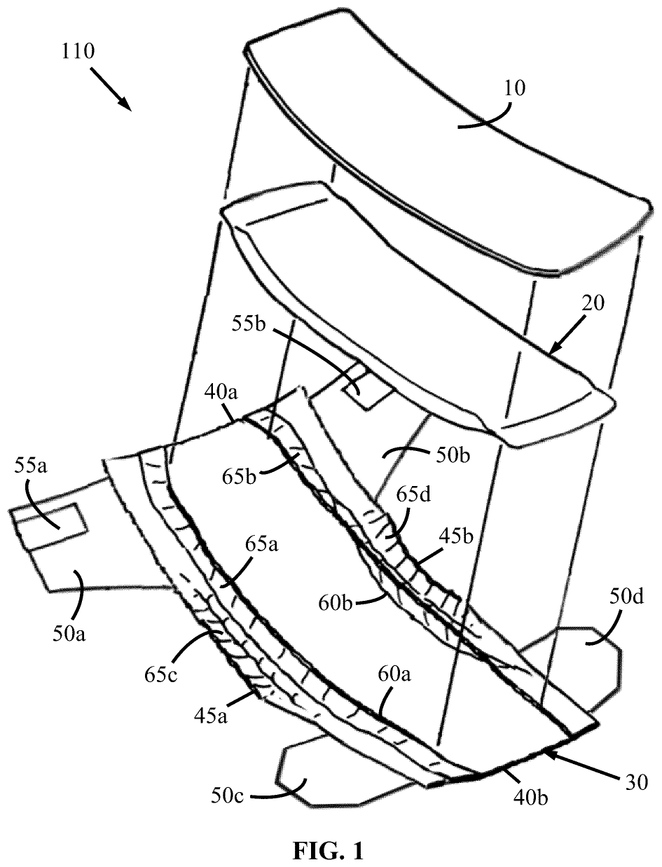

The embodiment of FIG. 1 depicts components of disposable absorbent article 110 that may comprise a topsheet 10, disposable absorbent insert 20, and backsheet 30. FIG. 1 shows insert 20 between topsheet 10 and backsheet 30. Liquid exudates are discharged onto and penetrate through topsheet 10 where they are absorbed by the insert 20, and the backsheet 30 prevents the exudates from leaking out of article 110. The waist-side edges 40a and 40b are the outermost edges along the width of article 110, and the leg-side edges 45a and 45b are the outermost edges along the length of article 110. Backsheet 30 includes fastening sections 50a and 50b that are attached on opposite ends to leg-side edges 45a and 45b proximate the waist-side edge 40a, and fastening sections 50c and 50d attached on opposite ends to leg-side edges 45a and 45b proximate waist-side edge 40b. Fasteners 55a and 55b are shown attached to fastening sections 50a and 50b to secure article 110 around the waist of the wearer. To enhance the containment and absorption of urine and other body exudates and provide improved leakage protection fluid barriers 60a and 60b may be utilized, and attached to backsheet 30 inward from and parallel to leg-side edges 45a and 45b. Elastic members 65a and 65b may be utilized with fluid barriers 60a and 60b, respectively. Elastic members 65c and 65d may be used to provide improved fit and to minimize the potential for leakage along the legs of the wearer.

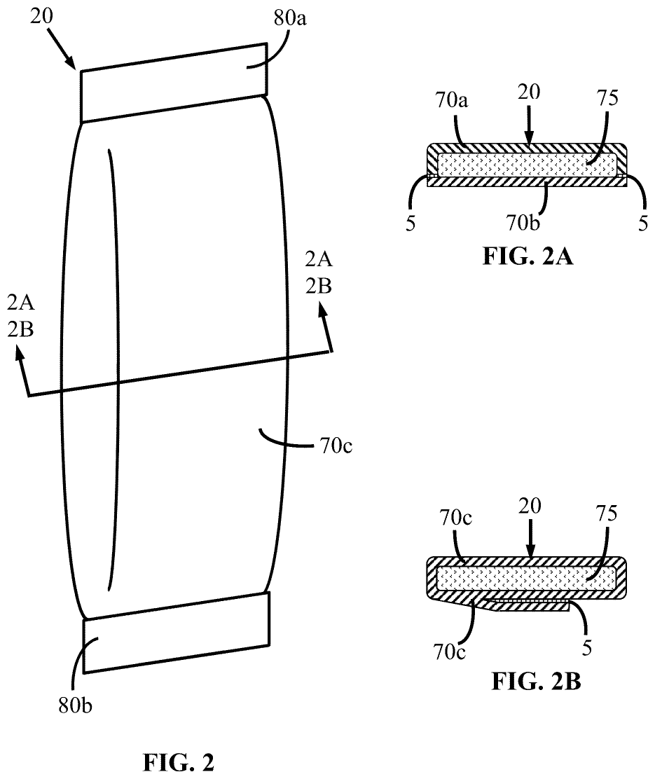

FIG. 2 is a perspective view of an embodiment of the disposable absorbent insert 20 that contains the absorbent contents of article 110. The insert 20 comprises the insert outer layer 70 that encases the absorbent insert inner layers 75 of article 110. Insert ends 80a and 80b are shown as the bonded edges of insert 20 that are adjacent to the waist-side edges 40a and 40b, respectively. Insert 20 is shown having a rectangular shape; although, it is readily apparent that other shapes may also be utilized based upon design requirements.

The embodiments of FIGS. 2A and 2B are cross-sectional views taken along lines 2A-2A and 2B-2B, respectively, of FIG. 2 showing the construction of insert 20. FIG. 2A shows that insert outer layers 70a and 70b may be joined using bonds 5 to encase insert inner layers 75 as shown in FIG. 2A. Optionally, an insert outer layer 70c may wrap around to encase the absorbent insert inner layers 75, and insert outer layer 70c may be attached using bonds 5 as shown in FIG. 2B.

Components of the article are joined using article bonds 5. Article bonds 5 comprises conventional methods and materials used in the art to join together adjacent layers and components. Sufficient examples for bonds 5 may comprise spraying adhesive on the whole or part of the surface, using a uniform continuous layer of adhesive, a patterned layer of adhesive, an array of separate lines or spots of adhesive, or a combination thereof. Alternatively, the attachment methods and materials may comprise using adhesives, heat bonds, pressure bonds, ultrasonic bonds, mechanical bonds, embossing, or any other suitable attachment methods or combinations of these attachment methods known to those in the art. The terms “join” or “bond” or “attach”, as used herein, are interchangeable and encompasses configurations whereby elements may be affixed directly or indirectly employing conventional techniques such as those well known in the art.

The liquid permeable disposable absorbent insert 20 refers to the element of the embodiments of articles 110, 120, 130, 140, and 150 having a substantial absorbent capacity. The insert 20 may comprise materials that are generally compressible, comfortable, non-irritating to the wearer's skin, and capable of absorbing and retaining liquids and certain body exudates. The total absorbent capacity and size of insert 20 is dictated by the absorbency requirements and can be varied to accommodate wearers ranging from infants through adults. Furthermore, the total absorbent capacity of insert 20 should be compatible with the design exudates loading in the intended use of the article. Absorbent insert 20 may be biodegradable and compostable.

The insert outer layer 70 may comprise materials ordinarily used in the art, and may have alternate embodiments comprising of insert outer layers 70a, 70b, and 70c. A suitable insert outer layer 70 that may be manufactured from a wide range of materials, such as natural fibers, synthetic fibers, or a combination of natural and synthetic fibers. For example, the insert outer layer 70 may be fabricated from a liquid pervious material, including but not limited to a nonwoven web or sheet of wet strength tissue paper, a spunbonded, meltblown or bonded-carded web, or a web of natural polymer filaments such as rayon, viscose, hemp, bamboo, or cotton.

Alternatively, insert outer layer 70 may be fabricated from a liquid permeable hydrophobic fibrous material that will allow liquid to readily wick moisture into the absorbent inner layers 75 and away from the wearer's skin. Insert outer layer 70 may be fabricated from a natural material that may be biodegradable and compostable that may breakdown over a relatively short period of time when exposed to natural elements such as air, heat and moisture that can accelerate the degrading process once discarded. Insert outer layer 70 contains and encases insert inner layers 75 of absorbent insert 20.

The absorbent insert 20 may comprise insert inner layers 75, or may comprise a combination of various absorbent layer materials superposed in facing relation, such as absorbent materials 75a (not shown) and dispersal layers 75b (not shown). Optionally, absorbent insert 20 may also include high-absorbency material 75c (not shown). Insert inner layers 75 may be biodegradable and compostable.

Insert inner layers 75 may be fabricated from absorbent material 75a that has some absorbency property or liquid retaining properties formed from materials that are generally compressible, comfortable, non-irritating to the wearer's skin, and capable of absorbing and retaining liquids and certain body exudates. Absorbent material 75a may be formed from materials ordinarily used in the art, and non-limiting examples may include wood pulp fluff, airfelt, airlaid, cellulose fiber, creped cellulose wadding, tissue, organic materials, or any other known absorbency materials or combinations of materials. Absorbent material 75a may be fluid permeable, biodegradable, and compostable.

The insert inner layers 75 may also include dispersal layer 75b that improves the tensile strength of absorbent insert 20 and reduces the tendency for it to split, lump or ball when wetted. Dispersal layer 75b improves lateral wicking of the absorbed exudates, thereby providing a more even distribution of absorbed human exudates throughout the absorbent insert 20. The materials used to form dispersal layer 75b may comprise materials ordinarily used in the art, for example, wet-strength cellulosic material, wet strength tissue paper, creped wadding, or the dispersal layer 75b may be omitted. Dispersal layer 75b may be attached to components of absorbent insert 20 as described for bonds 5. Dispersal layer 75b may be fluid permeable, biodegradable, and compostable.

High-absorbency material 75c may be employed to enhance absorption capacity and to reduce the overall size or thickness of the absorbent insert 20, and thereby improve wearer comfort and reduce the volume of disposable waste created by a soiled article. The high-absorbency material 75c used in the art is capable of absorbing substantially many times its weight in water. The high-absorbency material 75c may comprise materials ordinarily used in the art that comprise compounds to increase the absorbency of the body. Sufficient high-absorbency materials 75c may also be formed of natural materials such as plant-based materials that are used in the art, synthetic materials such as super-absorbent polymers, or a combination of natural and synthetic materials. The high-absorbency materials 75c may also be formed of biodegradable and compostable materials formed from degradable substances. High-absorbency material 75c may comprise a discrete layer separate from the absorbent materials 75a and the dispersal layers 75b. Alternatively, high-absorbency material 75c may be combined to be integral with absorbent materials 75a, or high-absorbency material 75c may be omitted entirely.

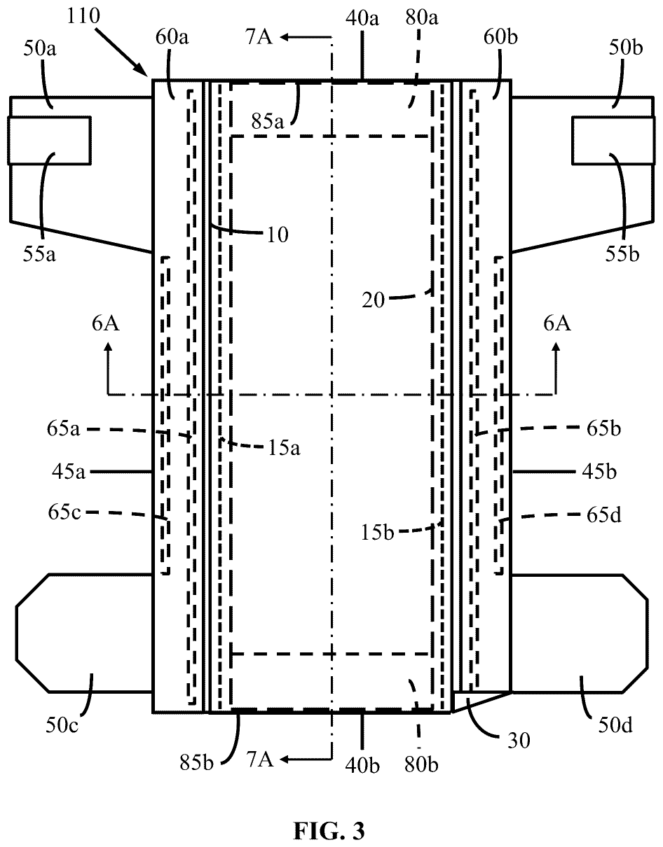

FIG. 3 includes components depicted in FIG. 1 for article 110. FIG. 3 shows the interior construction of article 110 depicted by hidden lines for the insert 20 and the elastic members 65a, 65b, 65c, and 65d, as well as a corner folded back to show the backsheet 30. FIG. 3 shows that topsheet 10 is attached to backsheet 30 using topsheet bonds 15a and 15b that are placed outward of insert 20.

Topsheet bonds 15a and 15b use a method or material for bonds 5 to join topsheet 10 and backsheet 30. Alternatively, topsheet bonds 15a and 15b may comprise a method or material used for bonds 5 that is a breakable bond to allow the user to detach topsheet 10 from backsheet 30 rather easily when pulled. Configurations and placements of topsheet bonds 15a and 15b may vary, with non-limiting examples including a patterned layer of adhesive, an array of separate lines or spots of adhesive, or a combination thereof.

Topsheet 10 is generally the wearer-facing surface that may also be the wearer-contacting surface. It may be desired that material forming topsheet 10 may be compliant, soft feeling, and nonirritating to the wearer's skin. The topsheet 10 may be a flexible, porous sheet which is liquid permeable permitting liquids to readily penetrate through its thickness. The topsheet 10 may be formed of a material may have a pore size that readily allows the passage of liquids, such as urine. Topsheet 10 may be fabricated from with a material having a tensile strength that is strong enough not to tear when the article is worn and wetted by the wearer and that maintains its strength during use. Topsheet 10 may be biodegradable and compostable to breakdown over a relatively short period of time once discarded after use, and when dispersed in water may disintegrate in the sewer or septic system. A suitable topsheet 10 may be manufactured from a wide range of materials or a combination of materials ordinarily used in the art, including but not limited to organic materials, natural filaments, plant-based fibers, cellulose, wood pulp, rayon, viscose, cotton, hemp, bamboo, wet strength tissue, a spunlace nonwoven sheet, or the like. Topsheet 10 may be fabricated from a nonwoven, spunbonded, meltblown or bonded-carded web.

Backsheet 30 is generally the garment-facing surface that may also be the garment-contacting surface. It may be desired that backsheet 30 may be formed of a thin, flexible, compliant, and liquid impermeable material that prevents the exudates absorbed and contained therein from wicking through to the clothing, bed sheet, and/or the environment of the wearer. Backsheet 30 may comprise materials ordinarily used in the art, for example, a web or sheet of plastic film such as thermoplastic film, or other suitable materials. Alternatively, the backsheet 30 may comprise a nonwoven, fibrous web which has been suitably constructed and arranged to be substantially liquid impermeable. Other suitable materials may include breathable materials which permit vapors to escape while still preventing exudates from passing through the backsheet 30. Backsheet 30 may be fabricated from one or more layers of materials used in the art. Backsheet 30 may be substantially opaque or transparent, and may have an embossed or matte surface. Backsheet 30 material may be recyclable.

In some circumstances it may be desired to include fastening sections 50a, 50b, 50c, and 50d formed of a material integral to article 110, may comprise separate components attached to article 110, or may be omitted. Fastening sections 50a and 50b are attached on opposite ends to leg-side edges 45a and 45b, respectively, proximate waist-side edge 40a, and fastening sections 50c and 50d attached on opposite ends to leg-side edges 45a and 45b, respectively, proximate waist-side edge 40b, as shown in FIGS. 1, 3, 4, 5, 10, and 13. Fastening sections 50a, 50b, 50c, and 50d may comprise materials ordinarily used in the art, and may employ various shapes, sizes, configurations, or placements based upon design requirements, or may be omitted.

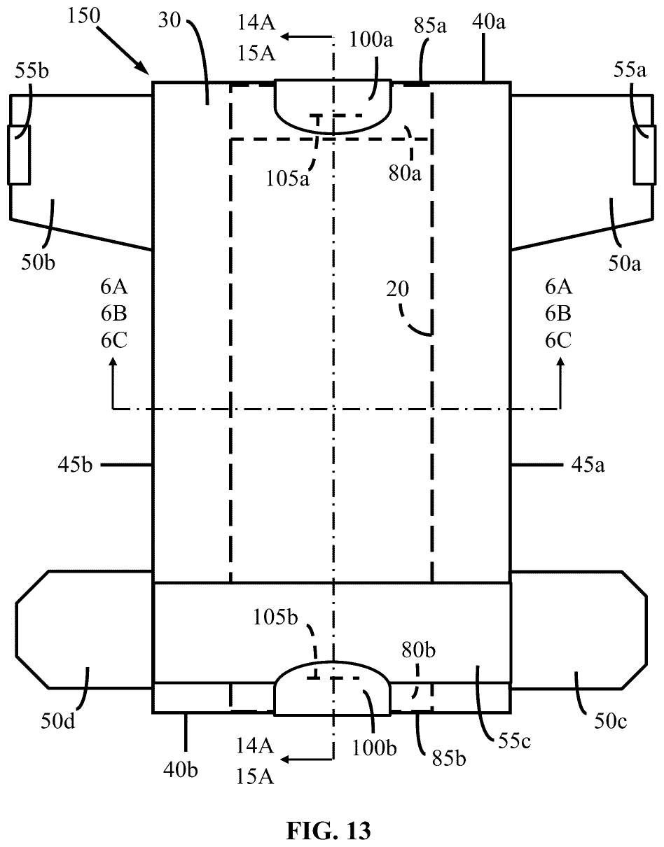

Fasteners 55a and 55b shown in FIGS. 1, 3, 4, 5, 10, and 13 may be attached to fastening sections 50a and 50b, respectively, using methods or materials for bonds 5. Fasteners 55a and 55b may comprise materials ordinarily used in the art. A fastener strip 5c (shown in FIG. 13) may comprise materials ordinarily used in the art, and may be bonded to the exterior side of backsheet 30 using bonds 5 proximate waist-side edge 40b. Fasteners 5a and 5b may be releasably attached to fastening strip 5c to secure the embodiments of articles 110, 120, 130, 140, and 150 around the waist of the wearer. Fasteners 55a and 55b may utilize methods or materials for bonds 5 to allow the user to detach fasteners 5a and 5b rather easily from fastener strip 5c.

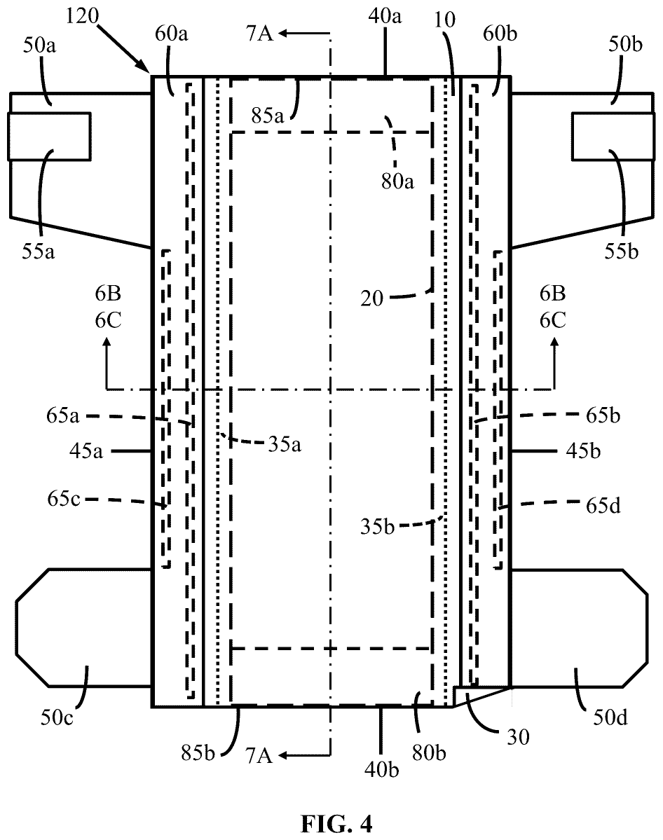

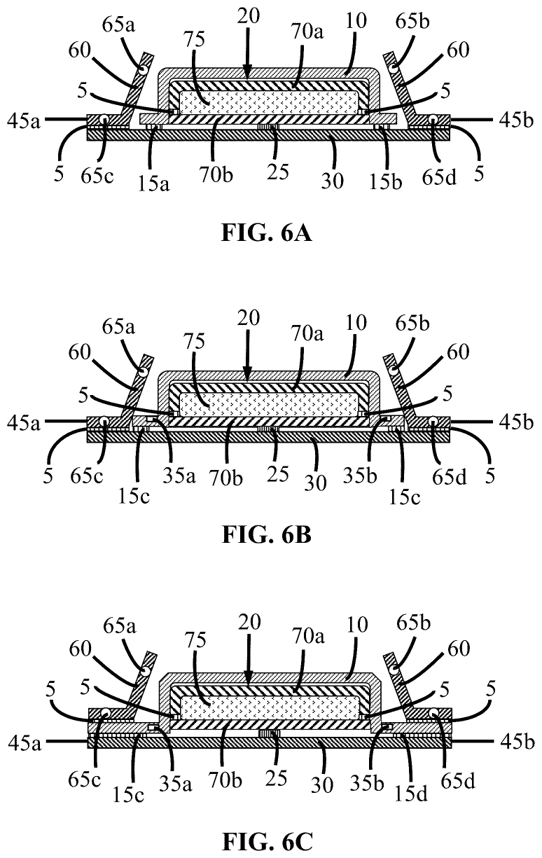

In some circumstances it may be desired that fluid barriers 60a and 60b may be utilized to enhance the containment and absorption of urine and other body exudates and provide improved leakage protection. Fluid barriers 60a and 60b may be attached to the interior side of backsheet 30 inward from and parallel to leg-side edges 45a and 45b, as shown in FIGS. 1, 3, 5, 10, and 13. Alternatively, fluid barriers 60 may be superimposed and attached throughout the length of the surface of topsheet 10 inward from and parallel to leg-side edges 45a and 45b as shown in FIGS. 4 and 6C. Fluid barriers 60 may comprise materials ordinarily used in the art.

Elastic members 65 may be utilized to provide improved fit and to conform to the shape of the wearer and minimize the potential for leakage. The elastic members 65 may comprise materials ordinarily used in the art. FIGS. 1, 3, 4, 5, 10, and 13 show elastic members 65a and 65b stretched along fluid barriers 60a and 60b, respectively, to provide improved leakage protection, and elastic member 65c and 65d are shown inward from leg-side edges 45a and 45b, respectively, to provide improved fit and to conform the shape of the wearer along the legs. Alternatively, elastic members 65 may also be attached to backsheet 30 and utilized proximate waist-side edges 40a and 40b to provide improved fit (not shown).

The insert ends 80a and 80b are integral extensions of insert 20 that are the bounded edges located proximate to the waist-side edges 40a and 40b, respectively. As a result of their distance from exudation points and their placements underneath topsheet 10, the insert ends 80a and 80b are less likely to be soiled after use. Either insert end 80a or 80b may be accessed from exposed edges 85a and 85b, respectively, for the user to pull insert 20.

The exposed edges 85a and 8b are located where the topsheet 10 is not bonded to the backsheet 30 to form an opening proximate waist-side edges 40a and 40b, respectively, to allow the user to access the insert ends 80a and 80b, insert protruding ends 90a and 90b, insert tabs 95a and 95b, or folded tabs 100a and 100b. Either exposed edges 85a or 85b, or both exposed edges 85a and 85b may be used with the embodiments of articles 110, 120, 130, 140, and 150.

FIG. 4 shows that article 120 includes all the components of article 110; however, the topsheet 10 is shown attached to backsheet 30 outward of insert 20 and substantially coterminous with waist-side edges 40a and 40b. Fluid barriers 60a and 60b may be attached to topsheet 10. The embodiment of FIG. 4 shows that along the surface of topsheet 10 may incorporate topsheet detachers 35a and 35b to facilitate separating the topsheet 10 from article 120. The detachers 35a and 35b are shown as two parallel lines inward of fluid barriers 60a and 60b and that may be incorporated along topsheet 10 throughout the length of article 10; although it is readily apparent that the detachers 35a and 35b may employ various configurations, shapes, sizes, or placements.

Topsheet 10 may incorporate topsheet detachers 35a and 35b therein to facilitate detaching the topsheet 10. The topsheet detachers 35a and 35b may be fabricated from a parallel lapped spunlace nonwoven fabric that can easily tear straight down the length of the article in a vertical direction. Topsheet detacher 35a and 35b may comprise perforations, lines of relative weakness, cuts, holes, openings, slits, notches, as well as areas on or attached to topsheet 10 that may be weakened by ultrasonics, embossing, adhesives, or other suitable means to open, tear, sever, separate or detach all or a substantial portion of topsheet 10 and that may be used to facilitate in detaching. A nonlimiting example of detacher 35a and 35b are shown in FIG. 4 that may comprise parallel lapped spunlace nonwoven fabric, or two parallel lines of relative weakness or a series of perforations inward from leg-side edges 45a and 45b that may be incorporated along topsheet 10 throughout the length of article. It is readily apparent that detacher 35 may employ various configurations, sizes, or placements. Although detachers 35a and 35b are shown in article 120 of FIG. 4, it is readily apparent that detachers 35a and 35b may also be used on the embodiments of articles 110, 130, 140, and 150.

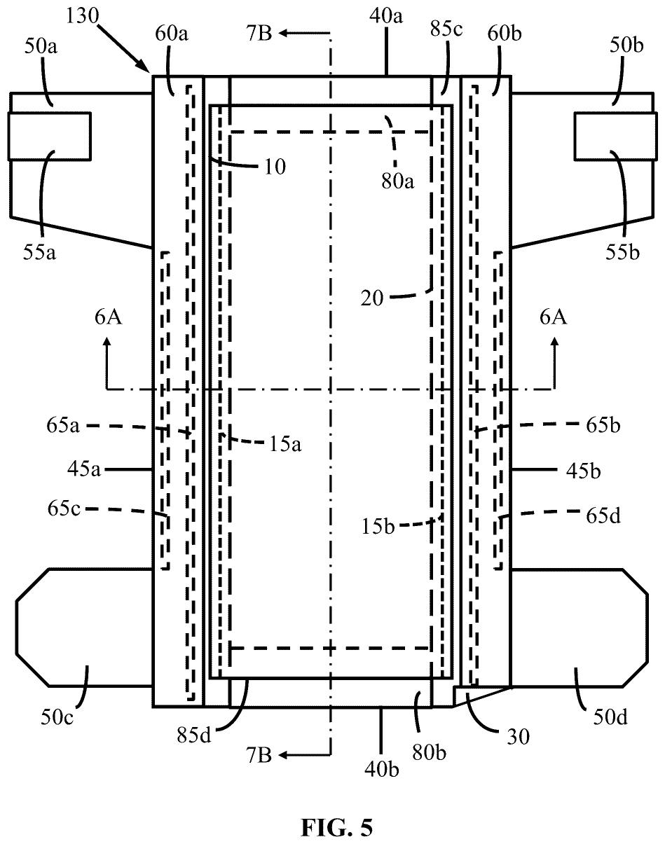

FIG. 5 shows that article 130 includes all the components of article 110; however, topsheet 10 is shifted inward from the waist-side edges 40a and 40b to expose insert ends 80a and 80b, and exposed edges 85c and 85d are shown outward of topsheet 10. Article 130 may use one exposed edge 85c or one exposed edge 85d, or may use both exposed edges 85c and 85d.

FIGS. 6A, 6B and 6C depict cross-sectional views along the centerlines of the width of articles 110, 120, 130, 140, and 150 to show the interior and exterior constructions of the various embodiments. FIGS. 6A, 6B, and 6C are shown having the construction of insert 20 depicted in FIG. 2A, and show insert 20 encased between topsheet 10 and backsheet 30; although, it is readily apparent that the construction of insert 20 depicted in FIG. 2B may be used, instead. Insert 20 is shown attached to backsheet 30 using insert-backsheet bond 25 that may comprise a method or material used for bonds 5 that is a breakable bond to allow the user to detach insert 20 from backsheet 30 rather easily when pulled. Configurations and placements of insert-backsheet bond 25 may vary, with non-limiting examples including a patterned layer of adhesive, an array of separate lines or spots of adhesive, or a combination thereof. FIGS. 6A and 6B show that fluid barriers 60a and 60b may be attached to backsheet 30 using a method or material for bonds 5, and FIG. 6C shows fluid barriers 60a and 60b may be attached to topsheet 10, as shown elastic members 65a and 65b may be attached to fluid barriers 60a and 60b, respectively. Elastic members 65c and 65d may be attached inward of leg-side edges 45a and 45b, respectively.

FIG. 6A is a cross-sectional view taken along lines 6A-6A of FIGS. 3, 5, 10, and 13 showing the construction of the embodiments of articles 110, 130, 140, and 150, respectively. Topsheet 10 may be attached to backsheet 30 outward of insert 20 and inward of the fluid barriers 60a and 60b using bonds 15a and 15b, respectively, that may comprise a method or material used for bonds 5 that is a breakable bond to allow the user to detach topsheet 10 from backsheet 30 rather easily when pulled.

FIG. 6B is a cross-sectional view taken along lines 6B-6B of FIGS. 4 and 13 showing the construction of the embodiments of articles 120 and 150, respectively. Topsheet 10 may be attached to backsheet 30 outward of insert 20 and inward of the fluid barriers 60a and 60b using bonds 15c and 15d that may comprise a method or material used for bonds 5. Topsheet 10 shows detachers 35a and 35b therein outward of insert 20. Alternatively, it is readily apparent detachers 35a and 35b and bonds 15c and 15d may be incorporated into articles of 110, 130, and 140.

FIG. 6C is a cross-sectional view taken along lines 6C-6C of FIGS. 4 and 13 showing the construction of the embodiments of articles 120 and 150, respectively, and is an alternate embodiment showing that topsheet 10 is attached to backsheet 30 using bonds 15c and 15d that may comprise a method or material used for bonds 5. Topsheet 10 is shown substantially coterminous with waist-side edges 40a and 40b, and topsheet 10 shows detachers 35a and 35b therein outward of insert 20. Alternatively, it is readily apparent that detachers 35a and 35b and bonds 15c and 15d may be incorporated into articles of 110, 130, and 140.

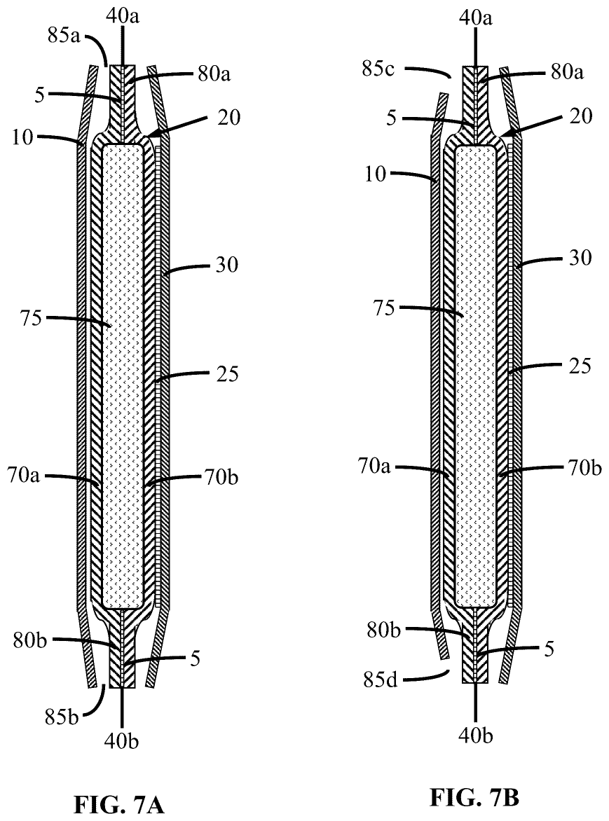

FIGS. 7A, 7B, 11A, 12A, 14A, and 15A depict cross-sectional views along the centerlines of the length of the embodiments of articles 110, 120, 130, 140, and 150 to show the interior and exterior constructions of the various embodiments. The various cross-sectional embodiments show that absorbent insert 20 is between topsheet 10 and backsheet 30, and that the insert 20 is attached to backsheet 30 using insert-backsheet bond 25 that may use a method or material for bonds 5 that may allow the user to detach the absorbent insert 20 from backsheet 30 rather easily when pulled. Proximate waist-side edges 40a and 40b, insert outer layers 70a and 70b may be joined using bonds 5 to encase insert inner layers 75.

FIG. 7A is a cross-sectional view taken along line 7A-7A of FIGS. 3 and 4, of articles 110 and 120, respectively. Topsheet 10, insert ends 80a and 80b, backsheet 30, and exposed edges 85a and 85b are shown to be substantially coterminous with waist-side edges 40a and 40b.

FIG. 7B is a cross-sectional view taken along line 7B-7B of FIG. 5, of articles 130. Backsheet 30 and insert ends 80a and 80b are shown to be substantially coterminous with the waist-side edges 40a and 40b. Topsheet 10 is shown shifted inward from waist-side edges 40a and 40b. Exposed edges 85c and 85d expose insert ends 80a and 80b outward of Topsheet 10. In use, topsheet 10 is the wearer-facing surface of the absorbent insert 20 where liquid exudates are discharged onto and penetrate through topsheet 10 to be absorbed by the absorbent insert 20. Backsheet 30 is the garment-facing surface that prevents the exudates absorbed and contained therein from wicking through.

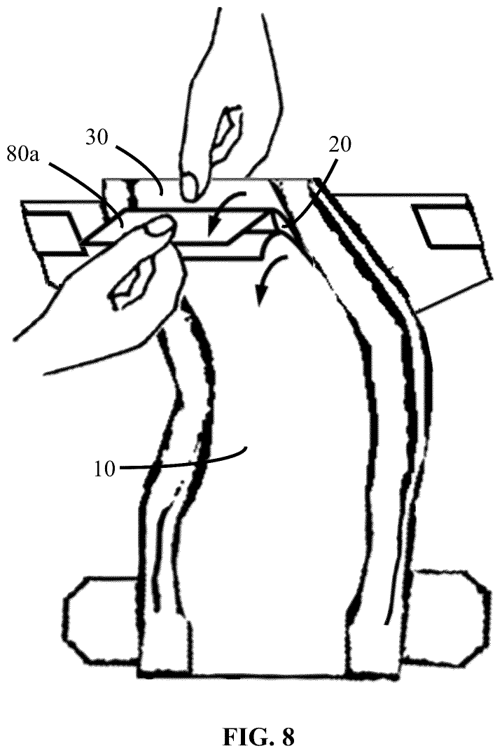

After use, one or both of insert ends 80a and 80b of article 110 may be accessed from exposed edge 85a or 85b, respectfully, to pull insert ends 80a and 80b. FIG. 8 shows the user pulling insert end 80a to pull insert 20 outwards to detach insert-backsheet bond 25 (not shown) that separates insert 20 from backsheet 30. As insert 20 is pulled away from backsheet 30 this causes topsheet 10 to be pushed outward from article 110 to detach releasable topsheet bonds 15a and 15b (not shown), and to detach topsheet 10 from backsheet 30 without the user having to directly touch, handle, or pull the topsheet 10, and without the need for the user to pull on any grips or tabs extending from or attached to topsheet 10. After use, the same method may be used to pull insert 20 outwards from the embodiments of articles 130, 140, and 150 to detach topsheet 10 from backsheet 30. Alternatively, detachers 35a and 35b (not shown) may be used to facility tearing topsheet 10 when insert 20 is pushed outward from articles 120 and 150.

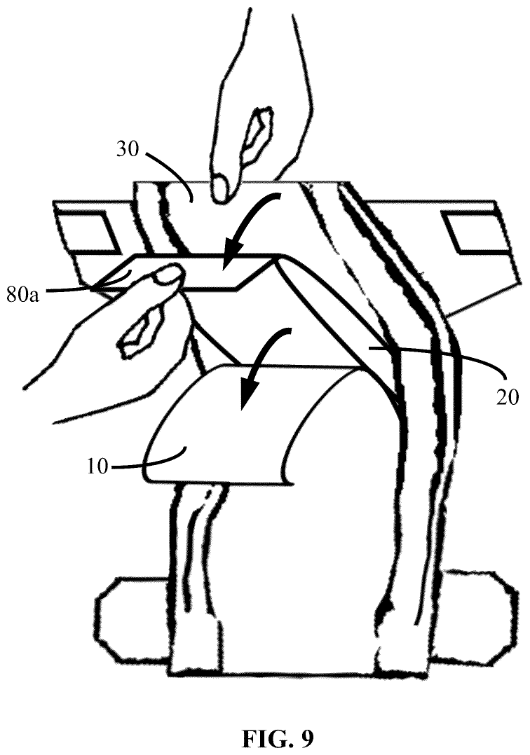

FIG. 9 shows that as the insert 20 is pulled out and separated further from backsheet 30, causes topsheet 10 to be detached more from the backsheet 30. As shown in FIG. 9, the topsheet 10 is being removed without the user having to touch or pull the topsheet 10 directly, and without any grips or tabs extending from or attached to topsheet 10 pulled by the user.

For disposal, once topsheet 10 has been completely removed from article 110, 120, 130, 140, or 150, topsheet 10 and exudates contained therein may be flushed down a toilet or septic tank. The biodegradable insert 20 may be completely separated from the plastic backsheet 30 to release the biodegradable material from the plastic material of the used disposable article to accelerate the degradation of the biodegradable material after use. Insert 20 may be discarded separately from backsheet 30. Insert 20 may be composted after use. Backsheet 30 may be recycled after use; it may be preferable for the backsheet 30 to be rinsed or wiped clean by the user to remove body exudates prior to the backsheet being recycled. Alternatively, insert 20 may remain attached to backsheet 30, so they may both be discarded in a landfill together after use.

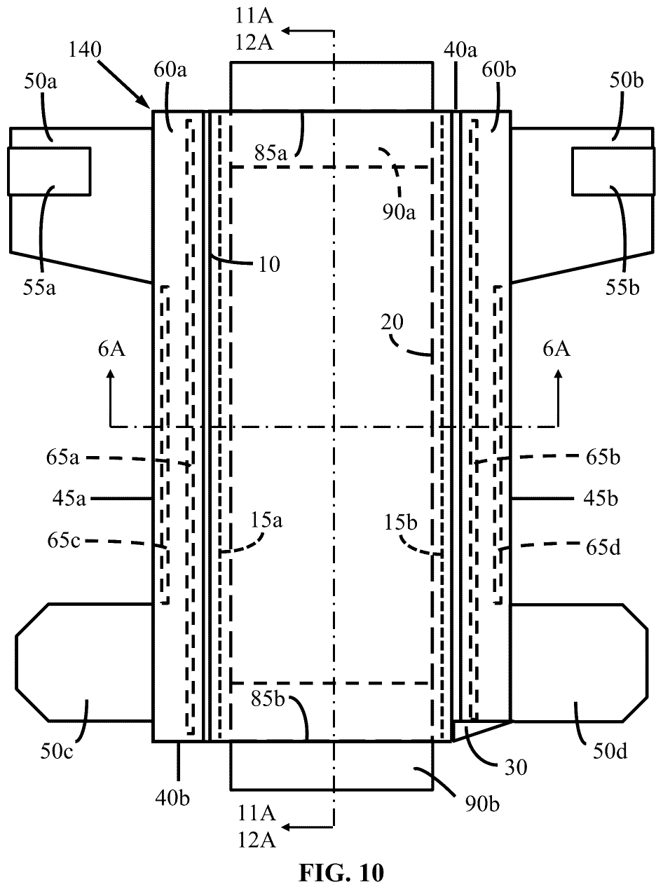

FIG. 10 shows that article 140 includes all the components of article 110; however, the insert protruding ends 90a and 90b are protruding out through openings of exposed edges 85a and 85b, respectively, and outward of waist-side edges 40a and 40b. Protruding insert tabs 95a and 95b may also be used and attached to insert ends 80a and 80b. Article 140 may use protruding end 90a or protruding end 90b, or may use both protruding ends 90a and 90b. As a result of their placements protruding outward from article 140 and their distance from exudation points, the protruding ends 90a and 90b are less likely to be soiled after use. After use protruding end 90a or protruding end 90b may be pulled by the user to detach the insert 20 from backsheet 30 to push topsheet 10 outward of article 140 and detach topsheet 10 as shown in FIGS. 8 and 9.

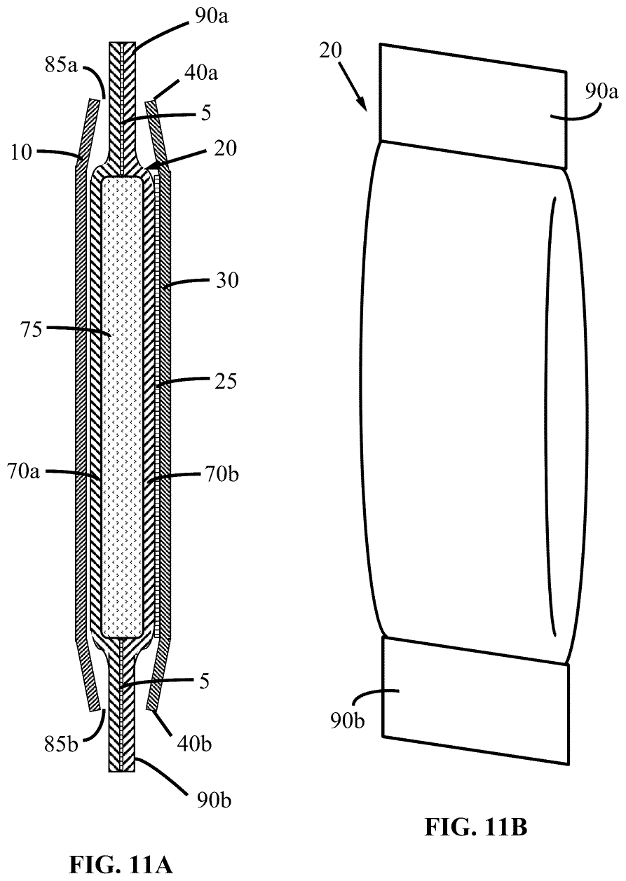

FIG. 11A is a cross-sectional view taken along line 11A-11A of FIG. 10 of article 140. Protruding ends 90a and 90b are protruding out through openings of exposed edges 85a and 85b and outward of waist-side edges 40a and 40b.

FIG. 11B is a perspective view of insert 20 of article 140. Insert 20 is shown with protruding ends 90a and 90b to provide reinforcement to pull insert 20.

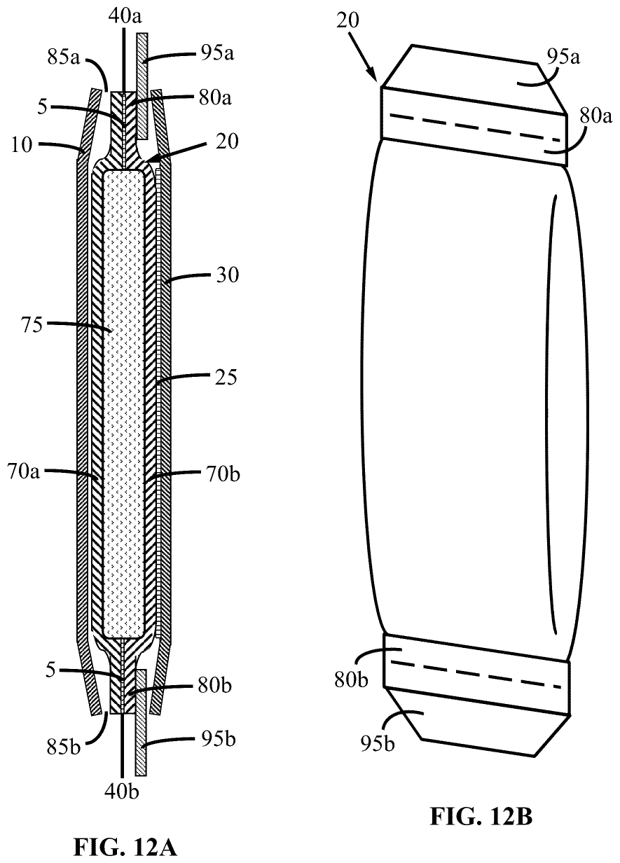

FIG. 12A is a cross-sectional view taken along line 12A-12A of FIG. 10 of article 140. Topsheet 10, backsheet 30, and insert ends 80a and 80b are shown to be substantially coterminous with the waist-side edges 40a and 40b. Protruding insert tabs 95a and 95b are attached to insert ends 80a and 80b, respectively, and may provide reinforcement to pull insert 20 to detach topsheet 10. Tabs 95a and 95b are protruding out through openings of exposed edges 85a and 85b and outward of waist-side edges 40a and 40b.

FIG. 12B is a perspective view of insert 20 of article 140. Insert 20 is shown with tabs 95a and 95b attached to insert ends 80a and 80b, respectively, to provide reinforcement to pull insert 20. Tabs 95a and 95b are shown with a trapezoidal shape to show that various shapes may be used.

FIG. 13 shows the exterior view that is the garment-side of article 150 which includes all the components of article 110; however, the folded tabs 100a and 100b are attached to insert ends 80a and 80b, respectively, and are protruding out through openings of exposed edges 85a and 85b, respectively, and outward of waist-side edges 40a and 40b. Folded tabs 100a and 100b are shown folded over backsheet 30 and are releasably attached to the exterior side of backsheet 30 with tab breakable bonds 105a and 105b; although, it is readily apparent that the protruding ends 90a and 90b of FIGS. 11A and 11B may also be used in place of insert ends 80a and 80b with folded tabs 100a and 100b omitted. Article 150 may include either folded tab 100a or folded tab 100b, or may use both folded tabs 100a and 100b. As a result of their placements protruding outward from article 150 and folded over to the exterior side of backsheet 30 and their distance from exudation points, the folded tabs 100a and 100b are less likely to be soiled after use. After use folded tab 100a or folded tab 100b may be pulled by the user to detach the insert 20 from backsheet 30 to push topsheet 10 outward and from article 140 and detach topsheet 10 as shown in FIGS. 8 and 9. Tab breakable bonds 105a and 105b may comprise a method or material used for bonds 5 that is a breakable bond to allow the user to detach folded tabs 100a and 100b from the exterior side of backsheet 30 rather easily when pulled.

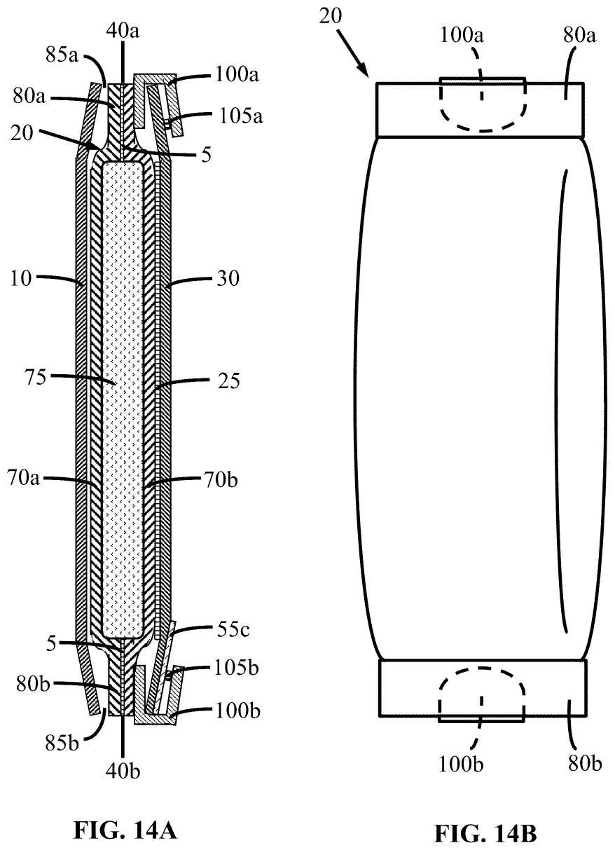

FIG. 14A is a cross-sectional view taken along line 14A-14A of FIG. 13 of article 150. Topsheet 10, insert ends 80a and 80b, and backsheet 30 are shown to be substantially coterminous with the waist-side edges 40a and 40b. Folded tabs 100a and 100b are attached to insert ends 80a and 80b, respectively. Folded tabs 100a and 100b are protruding out through openings of exposed edges 85a and 85b and outward of waist-side edges 40a and 40b. and are shown folded over backsheet 30 and releasably attached to the exterior side of backsheet 30 with tab breakable bonds 105a and 105b, respectively.

FIG. 14B is a perspective view of insert 20 of article 150. Insert 20 is shown with folded tabs 100a and 100b attached to insert ends 80a and 80b, respectively, to provide reinforcement to pull insert 20. Folded tabs 100a and 100b are shown with curved ends substantially shorter in width than the width of insert 20 to show that various shapes and sizes for tabs 100a and 100b may be used.

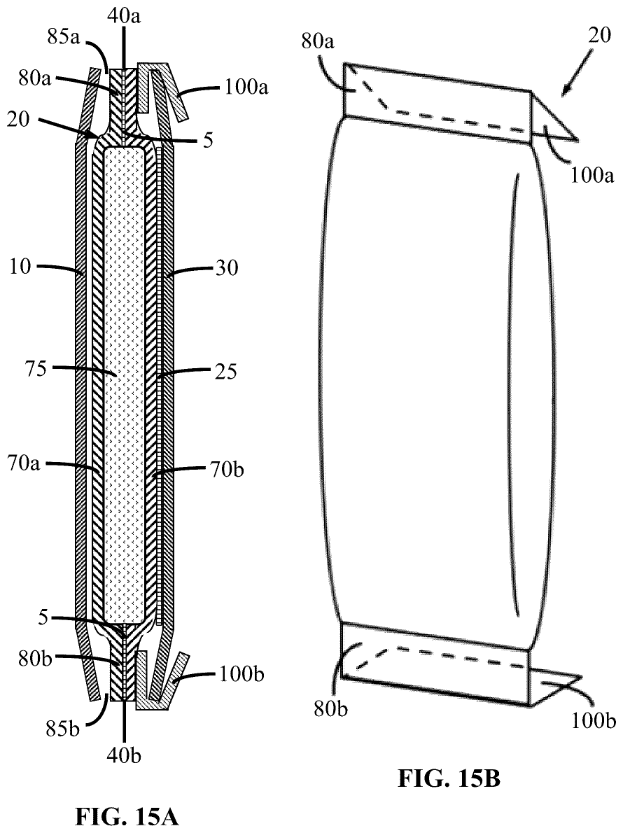

FIG. 15A is a cross-sectional view taken along line 15A-15A of FIG. 13 of article 150. Topsheet 10, insert ends 80a and 80b, and backsheet 30 are shown to be substantially coterminous with the waist-side edges 40a and 40b. Folded tabs 100a and 100b are attached to insert ends 80a and 80b, respectively, and folded over exterior side of backsheet 30. Folded tabs 100a and 100b protrude out through openings of exposed edges 85a and 85b and outward of waist-side edges 40a and 40b.

FIG. 15B is a perspective view of insert 20 of article 150. Insert 20 may use folded tabs 100a and 100b attached to insert ends 80a and 80b, respectively, to provide reinforcement to pull insert 20. Folded tabs 100a and 100b are shown to be rectangular in shape and coterminous width the width of insert 20 to show that various shapes and sizes for tabs 100a and 100b may be used.

### CONCLUSIONS AND RAMIFICATIONS

The detailed description of this disclosure has been made in the context of a disposable diaper article. It is readily apparent, that the absorbent structure of the present disclosure would also be suitable for other absorbent articles, such as, disposable inserts, disposable absorbent pads, disposable training pants, disposable incontinence articles, and the like.

The foregoing detailed description has been for the purpose of illustration. In the description, reference is made to the accompanying drawings which illustrate some embodiments of the disclosure. Variations in the operation and utilization of the disclosure are applicable. Also, alternative absorbent materials may be substituted for those described herein. Further, many of the particular aspects, features, or disposal options described in relation to one embodiment may be implemented in combination with aspects of other embodiments.

While particular embodiments of the present disclosure have been illustrated and described, these should not be construed as limiting the scope of the embodiments, but as merely providing illustrations of some embodiments. It would be obvious to those skilled in the art that the disclosure is capable of other and different embodiments, and that various other changes and modifications may be made without departing from the spirit and scope of the present disclosure. Accordingly, the scope of the disclosure should be determined not by the embodiments illustrated, but by the appended claims and their legal equivalents.

I claim:

1. A detachable disposable absorbent article, comprising:
a. a wearer-facing liquid permeable first layer;
b. a garment-facing second layer attached to the first layer, the first layer is unattached to the second layer along at least one waist-side edge to form an exposed edge; and
c. a liquid permeable disposable absorbent insert positioned between the first layer and the second layer, the insert is adapted to be releasably attached to the second layer, the insert is adapted to be accessed from said exposed edge and is adapted to be pulled outward to detach the insert from the second layer to cause the first layer to be pushed outward and detached from the article, whereby pulling the insert outward from the article and detaching the insert from the second layer is adapted to avoid directly handling and pulling the first layer when detaching the first layer from the article.

2. The article of claim 1, further comprising a breakable bond for releasably attaching the insert to the second layer.

3. The article of claim 1, further comprising a breakable bond for releasably attaching the first layer to the second layer.

4. The article of claim 1, further comprising a detacher for tearing the first layer.

5. The article of claim 1, further comprising a protruding end of the insert that protrudes outward of said exposed edge.

6. The article of claim 1, further comprising at least one tab attached to the insert that is adapted to provide reinforcement for pulling the insert.

7. The article of claim 1, wherein the first layer is adapted to be biodegradable and flushable.

8. The article of claim 1, wherein the insert is adapted to be biodegradable.

9. The article of claim 1, wherein the insert is adapted to be compostable.

10. The article of claim 1, wherein the second layer is adapted to be recyclable.

11. A detachable disposable absorbent article, comprising:
a. a wearer-facing liquid permeable first layer;
b. a garment-facing second layer attached to the first layer, the first layer is unattached to the second layer along at least one waist-side edge to form an exposed edge; and
c. a liquid permeable disposable absorbent insert positioned between the first layer and the second layer, the insert is adapted to be releasably attached to the second layer, the insert having at least one protruding end adapted to protrude out from said exposed edge and is adapted to be pulled outward to detach the insert from the second layer to cause the first layer to be pushed outward and detached from the article, whereby pulling the insert outward from the article and detaching the insert from the second layer is adapted to avoid directly handling and pulling the first layer when detaching the first layer from the article.

12. The article of claim 11, further comprising a breakable bond for releasably attaching the insert to the second layer.

13. The article of claim 11, further comprising a breakable bond for releasably attaching the first layer to the second layer.

14. The article of claim 11, further comprising a detacher for tearing the first layer.

15. The article of claim 11, wherein the protruding end is adapted to be folded over the waist-side edge to overhang onto the exterior side of the second layer, and wherein the protruding end is adapted to be releasably bonded to the exterior side of the second layer.

16. The article of claim 11, further comprising at least one tab attached to the insert that is adapted to provide reinforcement for pulling the insert.

17. The article of claim 11, wherein the first layer is adapted to be biodegradable and flushable.

18. The article of claim 11, wherein the insert is adapted to be biodegradable.

19. The article of claim 11, wherein the insert is adapted to be compostable.

20. The article of claim 11, wherein the second layer is adapted to be recyclable.
